(19) United States
(12) Patent Application Publication
(10) Pub. No.: US 2017/0215837 A1
(43) Pub. Date: Aug. 3, 2017

(54) ACOUSTIC SENSOR BASED GUIDEWIRE

(71) Applicant: BOSTON SCIENTIFIC LIMITED, Hamilton (BM)

(72) Inventors: VENKATESH HOSMANE RAMAKRISHNA, GURGAON (IN); AIDEN FLANAGAN, CO. GALWAY (IE); CHARUDATTA CHANDRAKANT ARADHYE, SOLAPUR (IN)

(73) Assignee: BOSTON SCIENTIFIC LIMITED, Hamilton (BM)

(21) Appl. No.: 15/418,213

(22) Filed: Jan. 27, 2017

Related U.S. Application Data
(60) Provisional application No. 62/288,900, filed on Jan. 29, 2016.

(51) Int. Cl. A61B 8/08; A61B 8/00; A61B 8/12; A61B 8/04; A61B 8/06

(52) U.S. Cl. CPC A61B 8/085; A61B 8/04; A61B 8/06; A61B 8/12; A61B 8/0891; A61B 8/4472; A61B 8/461; A61B 8/58; A61B 8/4483

(57) ABSTRACT

An intravascular medical device for characterizing a vascular occlusion is disclose The medical device may include an elongate shaft having a proximal end and a distal end and a tip coupled to the distal end of the shaft. An acoustic sensor may be coupled to the proximal end of the elongate shaft. The medical device may further include a signal processing system having a display screen and in communication with the acoustic sensor.

### CROSS-REFERENCE TO RELATED APPLICATIONS

This application claims the benefit of priority under 35 U.S.C. §119 to U.S. Provisional Application No. 62/288,900, filed Jan. 29, 2016, the entirety of which is incorporated herein by reference.

### TECHNICAL FIELD

The present disclosure pertains to medical devices, and methods for manufacturing medical devices. More particularly, the present disclosure pertains to guidewires for characterization of lesions (e.g. thrombus and/or plaques) and blood pressure sensing.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices.

In a first example, an intravascular medical device may comprise an elongate shaft having a proximal end and a distal end, a tip disposed at the distal end of the elongate shaft, a sensor disposed adjacent to the proximal end of the elongate shaft, and a signal processing system in communication with the sensor.

Alternatively or additionally to any of the examples above, in another example, the sensor may comprise an acoustic sensor, a micro-electromechanical systems acoustic pick up sensor, a contact microphone, a piezoelectric microphone, a haptic sensor, or combinations thereof.

Alternatively or additionally to any of the examples above, in another example, the sensor may be fixedly secured to the proximal end of the elongate shaft.

Alternatively or additionally to any of the examples above, in another example, the sensor may be releasably secured to the proximal end of the elongate shaft.

Alternatively or additionally to any of the examples above, in another example, the sensor may be magnetically coupled to the elongate shaft.

Alternatively or additionally to any of the examples above, in another example, the signal processing system may be magnetically coupled to the elongate shaft.

Alternatively or additionally to any of the examples above, in another example, the signal processing system further may comprise a display screen.

Alternatively or additionally to any of the examples above, in another example, the signal processing system may further comprise a calibration mode.

Alternatively or additionally to any of the examples above, in another example, the signal processing system may be in wireless communication with the sensor.

Alternatively or additionally to any of the examples above, in another example, the signal processing system may be configured to analyze one or more acoustic waveforms received at the sensor.

Alternatively or additionally to any of the examples above, in another example, the one or more acoustic waveforms may correspond to one or more characteristics of a vascular occlusion.

Alternatively or additionally to any of the examples above, in another example, the one or more acoustic waveforms may correspond to a fractional flow reserve (FFR) of a vessel.

In another example, a method for determining one or more characteristics of a vascular occlusion may comprise advancing a medical device through a vasculature of a patient to a location proximate an occlusion. The medical device may comprise an elongate shaft having a proximal end and a distal end, a tip disposed at the distal end of the elongate shaft, and an acoustic sensor disposed adjacent to the proximal end of the elongate shaft. The method may further comprise bringing the tip of the medical device into contact with the occlusion, receiving an acoustic waveform at the acoustic sensor, and translating the acoustic waveform to a characteristic of the occlusion.

Alternatively or additionally to any of the examples above, in another example, a signal processing system may translate the acoustic waveform and displays information regarding the characteristic of the occlusion on a display screen.

Alternatively or additionally to any of the examples above, in another example, bringing the tip of the medical device into contact with the occlusion may comprise repeatedly tapping the occlusion with the tip of the medical device.

In another example, an intravascular medical device may comprise an elongate shaft having a proximal end and a distal end, a tip disposed at the distal end of the elongate shaft, a sensor disposed adjacent to the proximal end of the elongate shaft, and a signal processing system in communication with the sensor.

Alternatively or additionally to any of the examples above, in another example, the sensor may comprise an acoustic sensor, a micro-electromechanical systems acoustic pick up sensor, a contact microphone, a piezoelectric microphone, a haptic sensor, or combinations thereof.

Alternatively or additionally to any of the examples above, in another example, the sensor may be fixedly secured to the proximal end of the elongate shaft.

Alternatively or additionally to any of the examples above, in another example, the sensor may be releasably secured to the proximal end of the elongate shaft.

Alternatively or additionally to any of the examples above, in another example, the sensor may be magnetically coupled to the elongate shaft.

Alternatively or additionally to any of the examples above, in another example, the signal processing system may be magnetically coupled to the elongate shaft.

Alternatively or additionally to any of the examples above, in another example, the signal processing system may further comprise a display screen.

Alternatively or additionally to any of the examples above, in another example, the signal processing system may further comprise a calibration mode.

Alternatively or additionally to any of the examples above, in another example, the signal processing system may be in wireless communication with the sensor.

Alternatively or additionally to any of the examples above, in another example, the signal processing system may be configured to analyze one or more acoustic waveforms received at the sensor.

Alternatively or additionally to any of the examples above, in another example, the one or more acoustic waveforms may correspond to one or more characteristics of a vascular occlusion.

Alternatively or additionally to any of the examples above, in another example, the one or more acoustic waveforms may correspond to a fractional flow reserve (FFR) of a vessel.

In another example, an intravascular medical device may comprise an elongate shaft having a proximal end and a distal end, a coil disposed over a length of the elongate shaft adjacent to the distal end thereof, a tip coupled to the distal end of the elongate shaft and a distal end of the coil, an acoustic sensor magnetically coupled to the proximal end of the elongate shaft, and a signal processing system having a display screen, the signal processing system in communication with the acoustic sensor.

Alternatively or additionally to any of the examples above, in another example, the signal processing system may be configured to analyze one or more acoustic waveforms received at the acoustic sensor.

Alternatively or additionally to any of the examples above, in another example, the one or more acoustic waveforms may correspond to one or more characteristics of a vascular occlusion.

Alternatively or additionally to any of the examples above, in another example, the one or more acoustic waveforms may correspond to the fractional flow reserve (FFR) of a vessel.

Alternatively or additionally to any of the examples above, in another example, the signal processing system may be releasably coupled to the proximal end of the elongate shaft.

In another example, a method for determining one or more characteristics of a vascular occlusion may comprise advancing a medical device through a vasculature of a patient to a location proximate an occlusion. The medical device may comprise an elongate shaft having a proximal end and a distal end, a tip disposed at the distal end of the elongate shaft, and an acoustic sensor disposed adjacent to the proximal end of the elongate shaft. The method may further comprise bringing the tip of the medical device into contact with the occlusion, receiving an acoustic waveform at the acoustic sensor, and translating the acoustic waveform to a characteristic of the occlusion.

Alternatively or additionally to any of the examples above, in another example, a signal processing system may translate the acoustic waveform and displays information regarding the characteristic of the occlusion on a display screen.

Alternatively or additionally to any of the examples above, in another example, bringing the tip of the guidewire into contact with the occlusion may comprise repeatedly tapping the occlusion with the tip of the medical device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of a portion of an example medical device.

FIG. 2 is a partial cross-sectional view of an example medical device disposed adjacent to an intravascular occlusion.

FIG. 3 is a partial cross-sectional view of an example medical device disposed at a first position adjacent to an intravascular occlusion.

FIG. 4 is a partial cross-sectional side view of a portion of another example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

During some endovascular procedures, characterization of the lesion is not discretely or objectively performed as a practice. The characterization of the lesion is typically performed by the “feel” of the lesion. For example, a physician may currently probe the lesion or thrombus with the distal tip of a guidewire, or other intravascular device.

The physician may then use the “feel” of the lesion (e.g. the perceived force of the impact) and the experience of the physician to determine the age of the thrombus. For example, a newer or fresh lesion may “feel” more squishy or jelly-like while an older lesion may “feel” harder or have less give. While angioscopy and/or intravascular ultrasound (IVUS) may provide information for making decisions regarding the appropriate treatment, they are not typically part of routine diagnostic workup as they may be cost prohibitive. It may be desirable to provide additional objective mechanisms for characterizing the age of a lesion to facilitate the physician in determining an appropriate treatment.

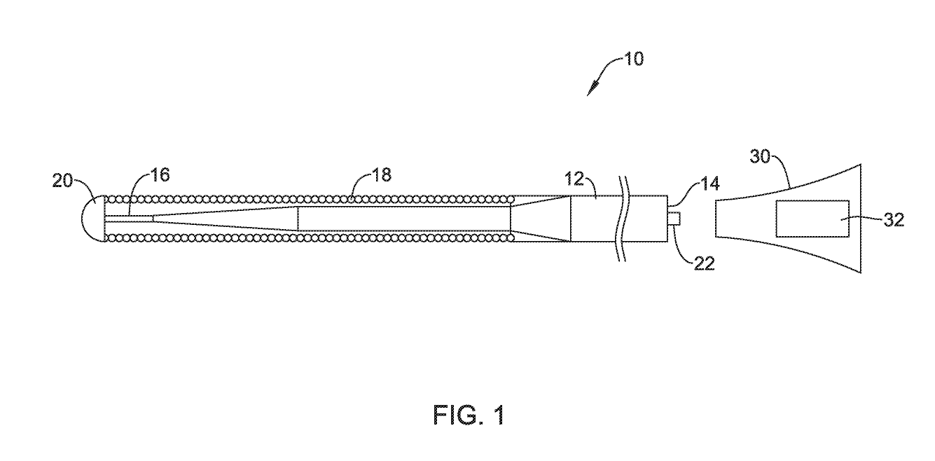

FIG. 1 illustrates a portion of an example medical device 10. In this example, medical device 10 is a guidewire 10. However, this is not intended to be limiting as other medical devices are contemplated including, for example, catheters, shafts, leads, wires, or the like. Guidewire 10 may include an elongate shaft or core wire 12 having a proximal end 14 configured to remain outside the body and a distal end 16. A coil 18 may be disposed over a length of the core wire 12 adjacent to the distal end 16. A tip 20 having a generally curved, atraumatic, shape, such as a solder tip, may be formed on the core wire 12 at or adjacent to the distal end 16. A portion of the coil 18 may be coupled to the tip 20. In some instances, a portion of the coil 18 may be embedded within the tip 20. Embedded is understood to be disposed within, coupled to, set in, implanted, fixed, etc. The tip 20 may, thus, fix the coil 18 relative to core wire 12. Alternatively, the coil 18 may be soldered to core wire 12 proximate to the tip 20. In some instances, the coil 18 may be replaced with a slotted tube or other flexible member.

The core wire 12 may be comprised of nickel-titanium alloy, stainless steel, a composite of nickel-titanium alloy and stainless steel, and/or include nickel-cobalt-chromium-molybdenum alloy (e.g., MP35-N). Alternatively, the core wire 12 may be comprised of metals, polymers, combinations or composites thereof, or other suitable materials. In some instances, a portion or all of the guidewire 10 may be radiopaque to allow the guidewire 10 to be viewed on a fluoroscopy screen, or other imaging technique, during a procedure. In some instances, the distal end 16 and/or coil 18 may be radiopaque to aid the physician in determining the location of the distal end 16 of the core wire 12.

The core wire 12 may be distally tapered. For example, the core wire 12 may include a plurality of distal segments or comprise a single, generally tapered distal end 16. Each distal segment may comprise a decreased outside diameter or individual segments may each taper along the length of a particular segment. A person of ordinary skill in the art could appreciate that a vast number of alternate configurations of segments and distal ends may be included without departing from the scope of the invention.

The guidewire 10 may include an acoustic sensor and/or microphone 22 attached to or adjacent to the proximal end 14 of the guidewire 10. While the sensor 22 is described as an acoustic sensor, it is contemplated that the sensor 22 may take the form of other sensors capable of providing information to the user, including, but not limited to, haptic sensors. For example, the acoustic sensor 22 may be attached to a side or end surface of the guidewire 10. In some embodiments, the acoustic sensor 22 may be positioned on a proximal end surface of the guidewire 10 extending generally orthogonal or transverse to a longitudinal axis of the guidewire 10. While the acoustic sensor 22 is described as attached to or positioned relative to the guidewire 10, it is contemplated that the acoustic sensor 22 may be attached to or formed as a unitary structure with other devices that may be used in combination with the guidewire. For example, the acoustic sensor 22 may be positioned on or formed with a hemostasis valve/port, an entry sheath, a guide catheter or any other device that allows for the detection of sound transmitted through the guidewire 10.

The acoustic sensor 22 may be a micro-electromechanical system (MEMS) acoustic pick up sensor. In other embodiments, the acoustic sensor 22 may be a contact or a piezoelectric microphone. These are just examples. The acoustic sensor 22 may take the form of any acoustic sensor and/or microphone desired, or combinations thereof. It is further contemplated that the sensor 22 may be a haptic sensor or an acoustic sensor or microphone used in combination with a haptic sensor. A haptic sensor may recreate the sense of touch (e.g. feel of the lesion) to the user by applying forces, vibrations, or motions to the user. In some instances, the acoustic sensor 22 may be releasably affixed or secured to the guidewire 10. For example, the acoustic sensor 22 may be magnetically coupled to the proximal end 14 of the guidewire 10. Other fixation mechanisms that do not distort the sound waves may also be used. Releasably securing the acoustic sensor 22 may allow the acoustic sensor 22 to be affixed to any guidewire (or other medical device) desired. In other instances, the acoustic sensor 22 may be permanently affixed or formed as a unitary structure with the guidewire 10. Placing the acoustic sensor 22 adjacent to the proximal end 14 of the guidewire 10 may allow the sensor 22 to be placed without requiring any modification to existing medical devices, however, it is contemplated that the acoustic sensor 22 may be positioned at any point desired along the length of the guidewire 10. As will be described in more detail below, the acoustic sensor 22 may be used to differentiate between different types of lesions based on the sounds received when the distal tip 20 of the guidewire 10 comes into contact with the lesion (e.g. different lesion types have different properties and thus may result in different sound profiles).

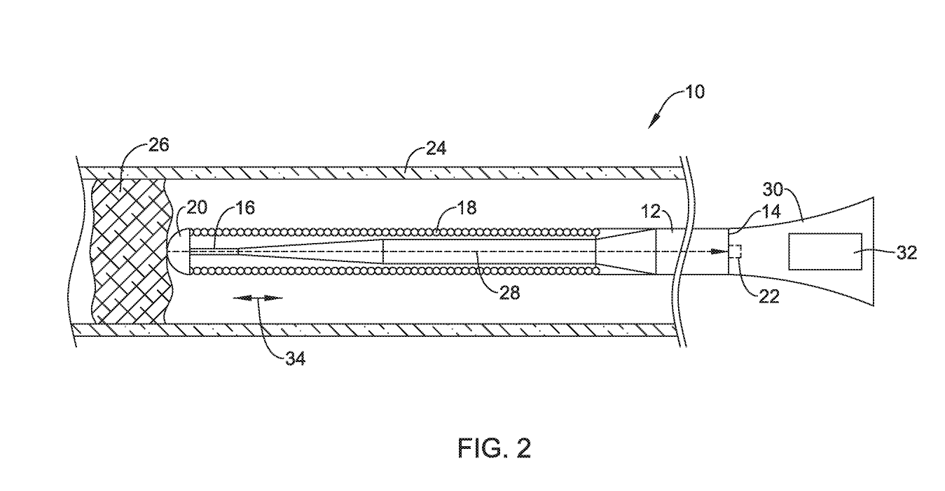

In use, a physician may use the guidewire 10 to characterize a lesion or thrombus. This may include advancing guidewire 10 through a blood vessel or body lumen 24 to a position that is proximal or upstream of an occlusion 26, as shown in FIG. 2. For example, the guidewire 10 may be advanced through a guide catheter (not explicitly shown) to a position adjacent to the occlusion 26. The physician may gently tap the distal tip 20 against the occlusion 26 to bring the tip 20 into contact with the occlusion 26. In some embodiments, the physician may repeatedly tap the distal tip 20 against the occlusion 26 to acquire multiple acoustic profiles. As the tip 20 contacts the occlusion 26 sound waves are generated and carried back to the acoustic sensor 22 through the guidewire 10, as shown schematically at 28. It is contemplated that occlusions having different textures may generate different sound or acoustic profiles upon contact between the distal tip 20 and the occlusion 26. This may allow the physician to determine the age of the occlusion, how hard or soft the occlusion is, how organized the occlusion is and/or is there is any underlying plaque. For example, an acute thrombus (e.g. recent thrombus) may provide a soft sound in contrast to a chronic thrombus which may provide a hard sound when the tip 20 is tapped against the occlusion 26. The presence of underlying plaque may also vary the frequency and/or amplitude of the sound waves thus allowing a physician to further characterize the occlusion. For example, an occlusion formed of an acute thrombus may provide a first sound waveform, an occlusion formed of an acute thrombus with underlying plaque may provide a second waveform, an occlusion formed of a chronic thrombus may provide a third waveform, and an occlusion formed of a chronic thrombus with underlying plaque may provide a fourth waveform. Each of these waveforms may be different from one another such that the occlusion 26 may be characterized. These are just examples. Other occlusion types are also contemplated. The physician may use the information obtained from the sound waveforms to determine the age and/or type of lesion and determine an appropriate treatment. In some instances, initial testing may be implemented with software such as LabVIEW DAQ systems. For example, illustrative sound profiles may be obtained and used for calibration and/or comparison purposes.

The guidewire 10 may further include a signal processing system 30. The signal processing system 30 may analyze the frequency and amplitude of the sound waves 28 and provide the physician with information regarding the occlusion 26 on a display 32. In some instances, the display 32 may provide alphanumeric information such as “hard” or “soft”. In other instances, the display 32 may provide a gradient color scale configured to indicate the age of the occlusion, or other characteristic. These are just examples. The signal processing system 30 may provide information to the physician in any manner desired.

The signal processing system 30 may be removably coupled to the proximal end 14 of the guidewire 10. FIG. 1 illustrates the signal processing system 30 uncoupled or disengaged from the guidewire 10 while FIG. 2 illustrates the signal processing system is 30 coupled to the guidewire 10. In some instances, the signal processing system 30 may be magnetically coupled to the guidewire 10. For example, the signal processing system 30 may have a first magnet configured to engage a second magnet in the guidewire 10. In some instances, the housing of the signal processing system 30 and/or the guidewire 10 may be formed of a magnetic material such additional magnets are not necessary to couple the guidewire 10 and the signal processing system 30. In other embodiments, the signal processing system 30 may be mechanically coupled to the guidewire 10 through, for example, a snap-fit, a press-fit, mating threads, etc. The acoustic sensor 22 may include electrical contacts configured to engage corresponding electrical contacts in the signal processing system 30 such that the acoustic signal 28 may be passed between the sensor 22 and the signal processing system 30. Alternatively, or additionally, the acoustic sensor 22 may be in wireless communication with the signal processing system 30. In other instances, the acoustic sensor 22 may be integrated with the signal processing system 30 thus forming a single component.

During some medical interventions, it may be desirable to measure and/or monitor the blood pressure within a blood vessel. For example, some medical devices may include pressure sensors that allow a clinician to monitor blood pressure. Such devices may be useful in determining fractional flow reserve (FFR), which may be understood as the ratio of the pressure after or distal of a stenosis (e.g., P_{d}) relative to the pressure before the stenosis and/or the aortic pressure (e.g., Pₐ). In other words, FFR may be understood as P_{d}/Pₐ.

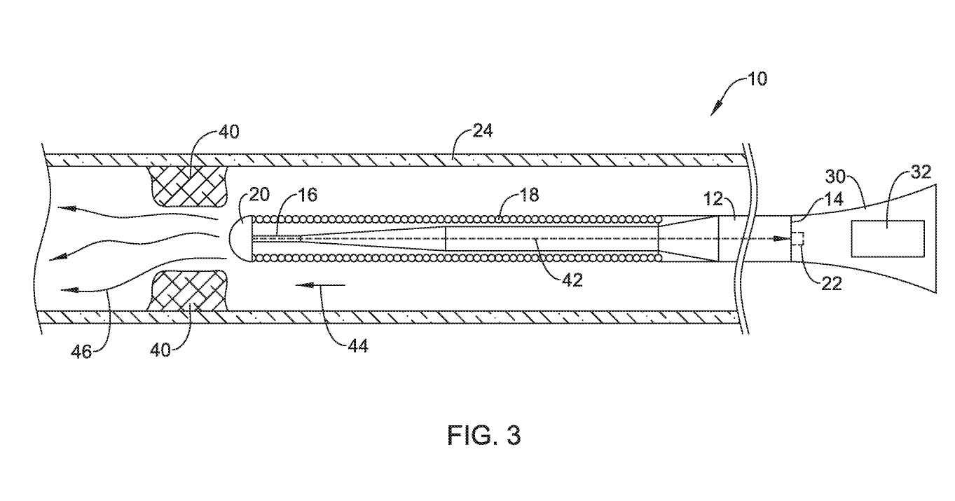

In some instances, the acoustic sensor 22 may be used to determine P_{d}/Pₐ, which will be described in detail with respect to FIG. 3. It is contemplated that the acoustic sensor 22 may be able to differentiate the turbulence 46 of blood distal to a lesion from the laminar flow 44 proximal to the lesion. For example, a lesion, such as lesion 40 illustrated in FIG. 3, may produce a noise called a bruit 42. The bruit may be generated by the turbulent flow 46 of blood distal to a lesion 40. The higher the pressure drop across the lesion 40, the higher the frequency components of the bruit will be. In some instances, the frequencies may generally be in the range of 50-400 Hertz (Hz), although it is contemplated that frequencies may be less than 50 Hz or greater than 400 Hz.

To determine the pressure drop across the lesion 40, the distal end 16/tip 20 of the guidewire 10 may be positioned proximal to the lesion 40, as shown in FIG. 3. The blood flow 44/46 may generate sound waves (e.g. the bruit) which are carried back to the acoustic sensor 22 through the guidewire 10, as shown schematically at 42. The frequency of the bruit 42 may be calculated by a Fourier transform algorithm. The bruit 42 may be dynamic, varying in both intensity and frequency with the heart's pulse. In some instances, an algorithm (which may be stored in a memory of the signal processing system 30) may analyze the frequencies at the same part of the pulse cycle. In other embodiments, the algorithm may build up a dynamic frequency profile across the pulse cycle that indicates the lesion's characteristics. It is contemplated that measuring the bruit 42 from within the vessel 24 may provide a greater signal to noise ratio than measuring the bruit 42 with a sensor on the skin. In some instances, initial testing may be implemented with software such as LabVIEW DAQ systems. For example, illustrative sound profiles may be obtained and used for calibration and/or comparison purposes.

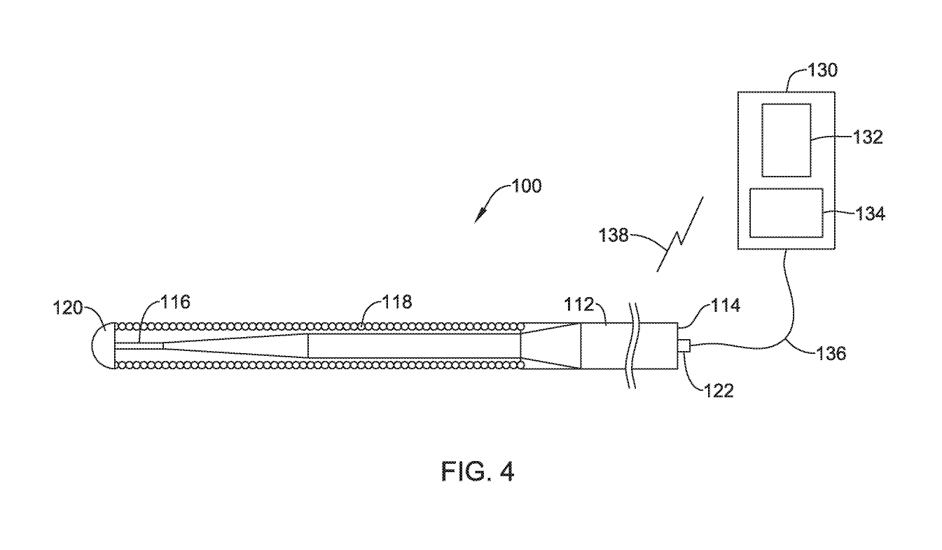

FIG. 4 illustrates a portion of another example medical device 100. In this example, medical device 100 is a guidewire 100. However, this is not intended to be limiting as other medical devices are contemplated including, for example, catheters, shafts, leads, wires, or the like. Guidewire 100 may be similar in form and function to guidewire 10 described above. Guidewire 100 may include an elongate shaft or core wire 112 having a proximal end 114 and a distal end 116. A coil 118 may be disposed over a length of the core wire 112 adjacent to the distal end 116. A tip 120, such as a solder tip, may be formed on the core wire 112 at or adjacent to the distal end 116. A portion of the coil 118 may be coupled to the tip 120. In some instances, a portion of the coil 118 may be embedded within the tip 120. Embedded is understood to be disposed within, coupled to, set in, implanted, fixed, etc. The tip 120 may, thus, fix the coil 118 relative to core wire 112. Alternatively, the coil 118 may be soldered to core wire 112 proximate to the tip 120. In some instances, the coil 118 may be replaced with a slotted tube or other flexible member.

The core wire 112 may be comprised of nickel-titanium alloy, stainless steel, a composite of nickel-titanium alloy and stainless steel, and/or include nickel-cobalt-chromium-molybdenum alloy (e.g., MP35-N). Alternatively, the core wire 112 may be comprised of metals, polymers, combinations or composites thereof, or other suitable materials. In some instances, a portion or all of the guidewire 100 may be radiopaque to allow the guidewire 100 to be viewed on a fluoroscopy screen, or other imaging technique, during a procedure. In some instances, the distal end 116 and/or coil 118 may be radiopaque to aid the physician in determining the location of the distal end 116 of the core wire 112.

The core wire 112 may be distally tapered. For example, the core wire 112 may include a plurality of distal segments or comprise a single, generally tapered distal end 116. Each distal segment may comprise a decreased outside diameter or individual segments may each taper along the length of a particular segment. A person of ordinary skill in the art could appreciate that a vast number of alternate configurations of segments and distal ends may be included without departing from the scope of the invention.

The guidewire 100 may include an acoustic sensor and/or microphone 122 attached to or adjacent to the proximal end 114 of the guidewire 100. The acoustic sensor 122 may be a micro-electromechanical system (MEMS) acoustic pick up sensor. This is just an example. The acoustic sensor 122 may take the form of any acoustic sensor and/or microphone desired. In some instances, the acoustic sensor 122 may be releasably affixed or secured to the guidewire 100. For example, the acoustic sensor 122 may be magnetically coupled to the proximal end 114 of the guidewire 100. Other fixation mechanisms that do not distort the sound waves may also be used. Releasably securing the acoustic sensor 122 may allow the acoustic sensor 122 to be affixed to any guidewire (or other medical device) desired. In other instances, the acoustic sensor 122 may be permanently affixed or formed as a unitary structure with the guidewire 100. Placing the acoustic sensor 122 adjacent to the proximal end 114 of the guidewire 100 may allow the sensor 122 to be placed without requiring any modification to existing medical devices, however, it is contemplated that the acoustic sensor 122 may be positioned at any point desired along the length of the guidewire 100. As above with respect to FIGS. 1-4, the acoustic sensor 122 may be used to differentiate between different types of lesions based on the sounds received when the distal tip 120 of the guidewire 100 comes into contact with the lesion (e.g. different lesion types have different properties and thus may result in different sound profiles) and/or FFR.

The guidewire 100 may further include a signal processing system 130 in either wired communication 136 or wireless communication 138 with the acoustic sensor 122. The signal processing system 130 may analyze the frequency and amplitude of the sounds waves and provide the physician with information regarding the occlusion on a display 132. In some instances, the display 132 may provide alphanumeric information such as “hard” or “soft”. In other instances, the display 132 may provide a gradient color scale configured to indicate the age of the occlusion, or other characteristic. These are just examples. The signal processing system 130 may provide information to the physician in any manner desired. The signal processing system 130 may also include a calibration button or mode 134. This may allow the user to initiate a calibration procedure with the guidewire system prior to use within a patient.

The signal processing system 130 may be provided as a separate unit from the guidewire 100. In some instances, the signal processing system 130 may be a stand-alone or dedicated system while in other instances the signal processing system 130 may be incorporated into other systems. For example, the signal processing system 130 may be incorporated into a fluoroscopy system or other computer system. In other embodiments, the signal processing system 130 may be incorporated into a mobile device such as a mobile phone or tablet computer.

The materials that can be used for the various components of guidewire 10 (and/or other guidewires disclosed herein) and the various tubular members disclosed herein may include those commonly associated with medical devices. For simplicity purposes, the following discussion makes reference to core wire 12 and other components of guidewire 10. However, this is not intended to limit the devices and methods described herein, as the discussion may be applied to other similar tubular members and/or components of tubular members or devices disclosed herein.

The various components of the devices/systems disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular medical device, comprising:
an elongate shaft having a proximal end and a distal end;
a tip disposed at the distal end of the elongate shaft;
a sensor disposed adjacent to the proximal end of the elongate shaft; and
a signal processing system in communication with the sensor.

2. The intravascular medical device of claim 1, wherein the sensor comprises an acoustic sensor, a micro-electromechanical systems acoustic pick up sensor, a contact microphone, a piezoelectric microphone, a haptic sensor, or combinations thereof

3. The intravascular medical device claim 1, wherein the sensor is fixedly secured to the proximal end of the elongate shaft.

4. The intravascular medical device of claim 1, wherein the sensor is releasably secured to the proximal end of the elongate shaft.

5. The intravascular medical device claim 4, wherein the sensor is magnetically coupled to the elongate shaft.

6. The intravascular medical device claim 1, wherein the signal processing system is magnetically coupled to the elongate shaft.

7. The intravascular medical device claim 1, wherein the signal processing system further comprises a display screen.

8. The intravascular medical device of claim 1, wherein the signal processing system further comprises a calibration mode.

9. The intravascular medical device of claim 1, wherein the signal processing system is in wireless communication with the sensor.

10. The intravascular medical device of claim 1, wherein the signal processing system is configured to analyze one or more acoustic waveforms received at the sensor.

11. The intravascular medical device of claim 10, wherein the one or more acoustic waveforms corresponds to one or more characteristics of a vascular occlusion.

12. The intravascular medical device of claim 10, wherein the one or more acoustic waveforms corresponds to a fractional flow reserve (FFR) of a vessel.

13. An intravascular medical device, comprising:
an elongate shaft having a proximal end and a distal end;
a coil disposed over a length of the elongate shaft adjacent to the distal end thereof;
a tip coupled to the distal end of the elongate shaft and a distal end of the coil;
an acoustic sensor magnetically coupled to the proximal end of the elongate shaft; and
a signal processing system having a display screen, the signal processing system in communication with the acoustic sensor.

14. The intravascular medical device of claim 13, wherein the signal processing system is configured to analyze one or more acoustic waveforms received at the acoustic sensor.

15. The intravascular medical device of claim 14, wherein the one or more acoustic waveforms corresponds to one or more characteristics of a vascular occlusion.

16. The intravascular medical device of claim 14, wherein the one or more acoustic waveforms corresponds to the fractional flow reserve (FFR) of a vessel.

17. The intravascular medical device of claim 14, wherein the signal processing system is releasably coupled to the proximal end of the elongate shaft.

18. A method for determining one or more characteristics of a vascular occlusion, the method comprising:
advancing a medical device through a vasculature of a patient to a location proximate an occlusion, the medical device comprising:
an elongate shaft having a proximal end and a distal end;
a tip disposed at the distal end of the elongate shaft; and
an acoustic sensor disposed adjacent to the proximal end of the elongate shaft;
bringing the tip of the medical device into contact with the occlusion;
receiving an acoustic waveform at the acoustic sensor; and
translating the acoustic waveform to a characteristic of the occlusion.

19. The method of claim 18, wherein a signal processing system translates the acoustic waveform and displays information regarding the characteristic of the occlusion on a display screen.

20. The method of claim 18, wherein bringing the tip of the guidewire into contact with the occlusion comprises repeatedly tapping the occlusion with the tip of the medical device.
